(19) United States
(12) Patent Application Publication
(10) Pub. No.: US 2020/0353111 A1
(43) Pub. Date: Nov. 12, 2020

(54) Arrangement for Storing and Disinfecting Toilet Brushes

(71) Applicants: Jochen Schmiddem, Berlin (DE); Saschinka Tillner_Schmiddern, Berlin (DE)

(72) Inventors: Jochen Schmiddem, Berlin (DE); Saschinka Tillner_Schmiddern, Berlin (DE)

(21) Appl. No.: 16/762,099

(22) PCT Filed: Nov. 2, 2018
(86) PCT No.: PCT/EP2018/079991

§ 371 Date: May 6, 2020

(30) Foreign Application Priority Data
Nov. 6, 2017 (DE) 10 2017 125 850.7
May 7, 2018 (DE) 10 2018 110 887.7

(51) Int. Cl. A61L 2/10; A61L 2/24; A46B 17/06; B08B 7/00; A47K 17/00

(52) U.S. Cl. CPC A61L 2/10; A61L 2/24; A46B 17/065; B08B 7/0057; A46B 2200/304; A61L 2202/122; A61L 2202/121; A61L 2202/11; A61L 2202/14; A47K 17/00

(57) ABSTRACT

Assembly for storing and disinfecting toilet brushes comprising a container or a housing for accommodating the entire toilet brush, wherein the container or the housing is provided with an opening for taking up the toilet brush; a lid, a flap or another closing portion for closing the opening; a UV-light source for exposing at least the handle of the toilet brush to UV-light the UV light source having a wavelength range and an intensity which is suitable for the disinfection of the exposed surfaces; is characterized in that means for opening the container or the housing without hand contact; and control means for controlling the UV-light source in such a way that the UV-light source emits UV-light only when the container or the housing is closed.

### TECHNICAL FIELD

The invention relates to an assembly for storing and disinfecting toilet brushes comprising
(a) a container or a housing for accommodating the entire toilet brush, wherein the container or the housing is provided with an opening for taking up the toilet brush;
(a) a lid, a flap or another closing portion for closing the opening;
(c) a UV-light source for exposing at least the handle of the toilet brush to UV-light the UV light source having a wavelength range and an intensity which is suitable for the disinfection of the exposed surfaces.

Toilet brushes serve to clean the inside of a toilet after its use. They are usually provided in the form of a simple round brush in the immediate environment of a toilet. The brush is used many times. Normally the brushes consist of plastic material and must be regularly disinfected and replaced. Still, a toilet brush is a continuous source of germs and shabby.

A particular problem is the transmission of germs through the handle of the toilet brush. The toilet brush is usually used after using the toilet but before washing the hands. This means that the handle of the toilet brush is taken with unwashed hands. Germs may be transmitted thereby. It is uncomfortable for the user to take up a handle by hand which has been used by possibly unknown people with unwashed hands.

### PRIOR ART

A large plurality of publications deals with the question of the hygiene of toilet brushes. The solutions suggested in patent literature involve the disinfection with liquid disinfectants, replaceable parts of the toilet brush and one-way brushes. None of the known solutions could convince the market because they are either difficult to manufacture or in practice not economic and/or ecologic. A plurality of known assemblies deals with the hygiene of the brush head, but not with the hygiene of the handle of the brush.

DE 20 2012 006 412 U1 discloses a holder for toilet brushes where stationary jets for spraying disinfectant onto the range of the handle of the brush. The assembly requires continuous re-filling of disinfectant. The handle is wet after disinfection. This is uncomfortable for the user. A sensor detects the inserting of the brush. This bears the risk that disinfectant is sprayed before the lid closes and exits the container.

US 2004/0159330 A1 discloses an assembly where the head of the brush but not the handle of the brush of a toilet brush is disinfected by means of UV radiation.

WO 2016/073831 A1 discloses the exposure of an entire toilet room with UV-radiation by means of an UV-radiator provided at the ceiling.

DE 20 2010 010 910 U1 discloses a device for providing a toilet brush with an disinfected handle. The assembly provides a disinfection chamber for the handle where there may be a UV lamp. The assembly has a complicated design and is not secured towards the outside against, for example, access by children. Only the handle is disinfected.

EP 0 356 896 B1 also discloses a device for disinfecting toilet instruments with ultraviolet radiation. The assembly is complex and difficult to handle.

Furthermore, it is known to disinfect tooth brushes with UV radiation. Contrary to tooth brushes toilet brushes are typically taken up by many different people with unwashed hands. Therefore, while the heads of tooth brushes are disinfected there is no requirement to disinfect the handle of a tooth brush.

US 2004/0089815 A1 and US 2004/0155201 A1 each disclose an assembly, where the head of a tooth brush is disinfected but not the handle. The container is closed by hand.

DE 196 14 779 A1 discloses a disinfection device for tooth brushes. The tooth brushes are exposed to UV light in a container. The head of the brush is arranged on the side of the UV light source. The UV light source can be manually switched on with a switch when the container is closed. Germs can be transferred with such an assembly through the switch.

U.S. Pat. No. 3,954,407 discloses a disinfection device for tooth brushes. Tooth brushes are circularly stuck into a holder around the UV light source. By manual rotation of the holder the UV light source is switched on and off. The assembly is not suitable for toilet brushes. Also, with this assembly germs may be transferred upon rotation of the holder.

### DISCLOSURE OF THE INVENTION

It is an object of the invention to provide a simple device of the above mentioned kind where at least the handle of the toilet brush is quickly and inexpensively disinfected and which is secured towards the outside at the same time.

According to the invention this object is achieved with a device of the above mentioned kind in that
(d) means for opening the container or the housing without hand contact; and
(e) control means for controlling the UV-light source in such a way that the UV-light source emits UV-light only when the container or the housing is closed.

The ultraviolet radiation emitted by the UV light source is provided for disinfection. Preferably, ultraviolet radiation of the short wavelength range is used, which is also called UVC radiation. The radiation has a relatively high intensity for good disinfection results. It is a disadvantage however, that such radiation is dangerous for skin, mucosa and eyes. Therefore, the invention provides that the exposure takes place only when the container is fully closed. The radiation is absorbed by the container and cannot exit outwards. It is a further advantage of the closed container that small children cannot easily access the toilet brush.

The container must be opened for the use of the toilet brush. For this purpose a contactless mechanism is provided which, in particular, must not be operated manually. The mechanism can be started electronically and activate, for example, by means of a motion sensor or a light barrier, a motor, a hydraulic mechanism or a pneumatic mechanism.

The mechanism, however, may also be mechanic. A particularly simple modification of the invention provides that the lid is connected to a pedal, by which the lid can be moved to an open position with the foot. Such a mechanism is known from bins already and does not provide a new challenge for the user. With such a mechanism also no manual contact to any of the portions of the assembly is necessary. The only thing which is touched by the user is the handle of the now disinfected toilet brush.

In a preferred embodiment of the invention it is provided that the container is provided with a releasable insert for the toilet brush. The insert can be easily removed and cleaned. The insert can be adapted to the form of the toilet brush. The container can be designed in an agreeable and stable manner without having to consider the shape of the toilet brush. The suitably shaped insert insert in turn can enable a good holding of the toilet brush and enable the release of dripping water. Also, it provides the possibility to exchange the insert independently of the remaining assembly. Precious materials may then be used for the container.

Preferably, the insert is provided with a bottom having a profile adapted to receive the toilet brush with a corresponding profile. For example, the profile can comprise a projection or a pin extending upwards in the centre. The toilet brush can be provided with an opening on its underside adapted to be stuck on the projection or pin. Thereby, the toilet brush is simultaneously held and centred whereby an even exposure is enabled.

A suitable toilet brush does not have brushes but stiff flags, such as made of silicon, provided at a shaft with a handle. The flags extend outwards in a radial direction. If the flags are designed projecting in the range remote to the axis this will enable reaching the range below the toilet rim.

Otherwise the toilet brush has a round profile at its lower end corresponding to the profile of typical toilet brushes. The use of silicon flags has the advantage that water repellent features cause a good cleaning effect but little dripping water.

In a particularly preferred embodiment of the invention it is provided that the UV-light source and the control means are accommodated in the container lid. The lid is part of the container and usually does not have any contact with the brush or water. In particular the lid can be provided with a further bottom whereby control means are additionally protected. The UV light can be directed directly or through a window with high UV transparency into the interior of the container. The window can be made of, for example, quartz or borosilicate.

In a particularly advantageous modification of the invention it is provided that the control means comprise a timer for determining the exposure time and the UV light source is switched off after a set exposure time. The exposure with UVC radiation can be, for example, carried out for a duration of 10 seconds to 2 minutes, such as 30 seconds. Such a duration is sufficient for a good disinfection. It is not necessary to activate the light source any further. It can be switched off after the set exposure time.

Preferably, it is provided that the light source operates without cables, such as with batteries or accumulators. No plug will then be necessary which normally is not present in the range of a toilet. The life span of the energy source can be prolonged by switching off after reaching a suitable exposure time.

In a further modification of the invention a sensor is provided for detecting the position of the container lid, the signals of such sensor being transmitted to the control means. In particular, it can be provided that the sensor is provided with a pin switch, a contact with book cover function or another contact, detecting when the container is closed. Thereby, the control means can generate a signal for starting the UV light. The start of the exposure time is when the lid is closed. The end is after a set or adjustable exposure time. If the sensor detects that the lid is closed the exposure is started. When the lid is opened the exposure is either interrupted or not started at all. With interrupted exposure this can either be continued or started from the beginning.

In a particularly advantageous modification of the invention it is provided that the sensor is integrated in a separate closing unit which also accommodates the hinge and possibly required further components and/or interfaces. The module can be fixed to the outside of the container or at the lid.

In a particularly advantageous modification of the invention it is provided that the insert is provided with a radially outwardly projecting upper rim for placing the insert in such a way on the edge of the container that it is at least partly visible from the outside even with closed lid. Thereby, the insert is held in its position. It can be seen from the outside if an insert is present in the container or not. Furthermore it can be provided that the insert is made of acryl or another material which will colourfully shine upon exposure to radiation of the UV-light source. It can then be recognized from the outside if there is exposure to radiation or not. This enables the user to wait before he takes up the toilet brush. The user can then be sure that at least the handle of the toilet brush is disinfected.

Further modifications of the invention are subject matter of the subclaims. An embodiment is described below in greater detail with reference to the accompanying drawings.

### BRIEF DESCRIPTION OF THE DRAWINGS

FIG. 1 is a longitudinal cross section of a toilet brush with silicon flags.

FIG. 2 is a top view on the toilet brush of FIG. 1.

FIG. 3 is a longitudinal cross section of an insert for a toilet brush made of acryl.

FIG. 4 is a top view on the assembly of FIG. 3.

FIG. 5A is a longitudinal cross section of a lid for a toilet brush in an insert in an exploded view.

FIG. 5B is a longitudinal cross section on the lid of FIG. 5A in an assembled state.

FIG. 6A is a top view on a container for a toilet brush and an insert.

FIG. 6B is a longitudinal cross section of the container of FIG. 6A.

FIG. 7 is a longitudinal cross section of the container of FIG. 5 with a bottom, closed lid and inserted insert and toilet brush.

FIG. 8 is a longitudinal cross section of the container in FIG. 7, but with an open lid.

FIG. 9 is a top view on the container of FIG. 7 with a base plate and a pedal.

FIG. 10 is a front view of the container of FIG. 9 in the direction of the pedal.

FIG. 11 is a side view of the container of FIG. 10 in the direction perpendicular to the pedal with closed lid. FIG. 12 is a front view of the container of FIG. 10 in the direction perpendicular to the pedal with open lid.

FIG. 13 is a back view of the container of FIG. 10 with closed lid.

FIG. 14 is a perspective view of a container for a toilet brush suspended at a wall according to a second embodiment.

FIG. 15 is a side view of the container of FIG. 14 with closed lid.

FIG. 16 is a side view of the container of FIG. 14 with open lid in an assembled state.

FIG. 17 is a vertical cross section of the assembly of FIG. 15.

FIG. 18 is a vertical cross section of the assembly of FIG. 16.

FIG. 19 separately shows the lid for the assembly of FIG. 14 from below.

FIG. 20 separately shows the lid for the assembly of FIG. 14 from the side.

FIG. 21 is a cross sectional view of the lid for the assembly of FIG. 14 laterally to the rotational axis.

FIG. 22 separately shows the lid for the assembly of FIG. 14 from above.

FIG. 23 is an exploded view of the container of FIG. 14 with insert and brush.

FIG. 24 is a top view on the container of FIG. 14 from above.

FIG. 25 is a top view onto the insert for a container of FIG. 14 with insert and brush.

FIG. 26 is a perspective view of a housing for accommodating a toilet brush in an open state according to a third embodiment.

FIG. 27 shows the assembly of FIG. 26 from the side.

FIG. 28 shows the assembly of FIG. 26 from the front.

FIG. 29 shows the assembly of FIG. 26 as a perspective view in a closed state.

FIG. 30 is an exploded view of the assembly of FIG. 26.

FIG. 31 is a vertical cross section of the assembly of FIG. 26 along a cross sectional plane A-A in a closed state.

FIG. 32 is a vertical cross section of the assembly of FIG. 26 along a cross sectional plane B-B in a closed state.

FIG. 33 is a vertical cross section of the assembly of FIG. 26 along a cross sectional plane C-C in a closed state.

FIG. 34 is a vertical cross section of the assembly of FIG. 26 along a cross sectional plane D-D in a closed state.

FIG. 35 is a vertical cross section of the assembly of FIG. 26 A-A in an open state with inserted toilet brush and insert.

FIG. 36 is a vertical cross section of the assembly of FIG. 26 in an open state with inserted insert and illustrates how the toilet brush is taken up.

FIG. 37 is a vertical cross section of the assembly of FIG. 26 in an opened state with separate insert and toilet brush.

FIG. 38 illustrates the installation of the assembly of FIG. 26 at a wall.

FIG. 39 illustrates the flush-mounted installation of the assembly of FIG. 26.

FIG. 40 is a perspective view of the visible front side of the assembly of FIG. 26 in an open state.

FIG. 41 illustrates the installation of the assembly of FIG. 26 behind a revision or decorative plate next to the toilet.

FIG. 42 is an exploded view of the assembly of FIG. 26.

### DESCRIPTION OF THE EMBODIMENTS

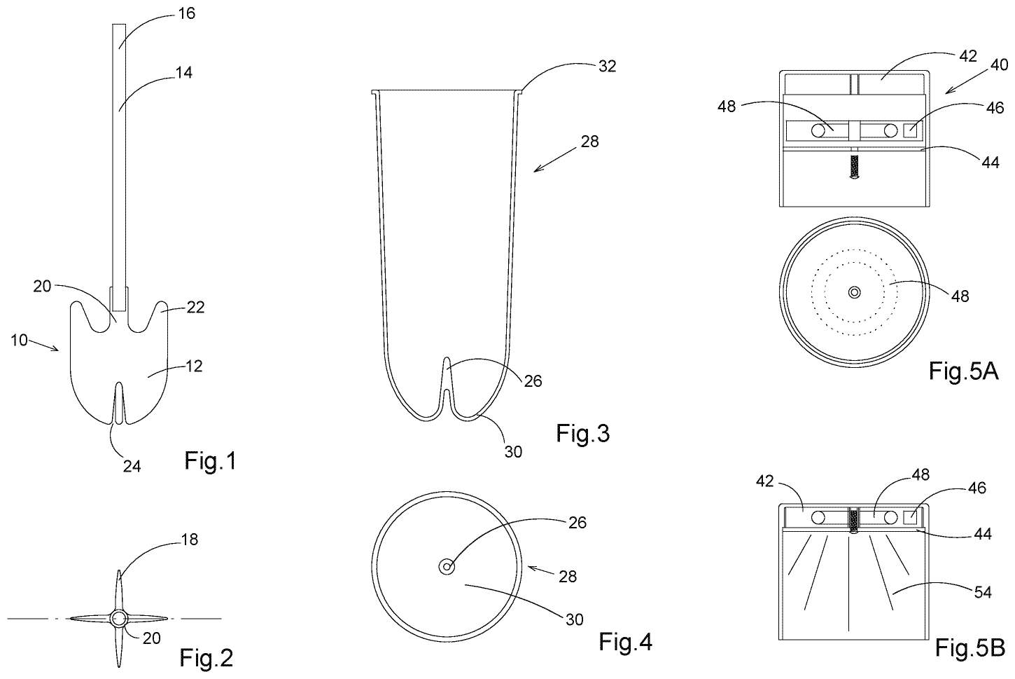

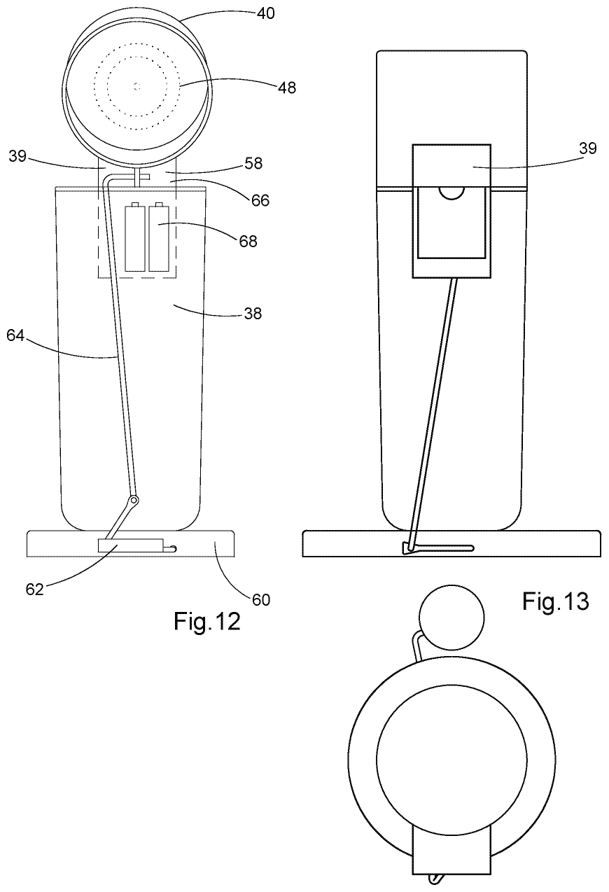

### 1. Embodiment (FIG. 1 to FIG. 13)

FIG. 1 shows a toilet brush generally designated with numeral 10. The toilet brush 10 has a brush head 12 and a shaft 14. The upper portion 16 of shaft 14 forms a handle. The shaft 14 is elongated and pin-shaped. The brush head 12 consists of silicon flags 18 which are shaped star-like and form an integral part of a ring 20 in the form of an annular receptacle. This can be well recognized in the top view of FIG. 2. The ring 20 is stuck onto the lower end of the shaft 14 and fixed thereto.

In the range remote to the axis of the brush head 12 the flags 18 slightly extend upwards with a small extension 22 beyond the remaining part of the brush head. This can be recognized in FIG. 1. With the extension the range below the toilet rim can be reached and cleaned also. On the underside the brush head 12 forms a groove 24. With the groove 24 the toilet brush 10 can be stuck onto an upwardly extending pin 26 in an insert 28 and centred in the insert 28. The insert 28 is separately shown in FIGS. 3 and 4.

The insert 28 serves to accommodate the toilet brush 10 when it is not used for cleaning. The insert 28 of the present embodiment consists of acryl. It is understood that other materials may also be used which have different or similar characteristics. The insert 28 is essentially cylindrical. The bottom of the insert is slightly curved and the pin 26 is shaped in such a way that the toilet brush 10 will not fully reach the bottom 30. This can be recognized in FIG. 7. If dripping water 13 accumulates at the bottom of the insert 28 it is avoided thereby that the toilet brush 10 is unnecessarily contacted therewith.

At its upper end the insert 28 forms a radially outwardly projecting rim 32. With the rim 32 the insert 28 lays on the upper edge 36 of an outer container 38. This can be recognized in FIG. 7.

The rim 32 of the insert 28 is aligned with the outer wall of the container 38 and, thereby, visible from the outside at all times.

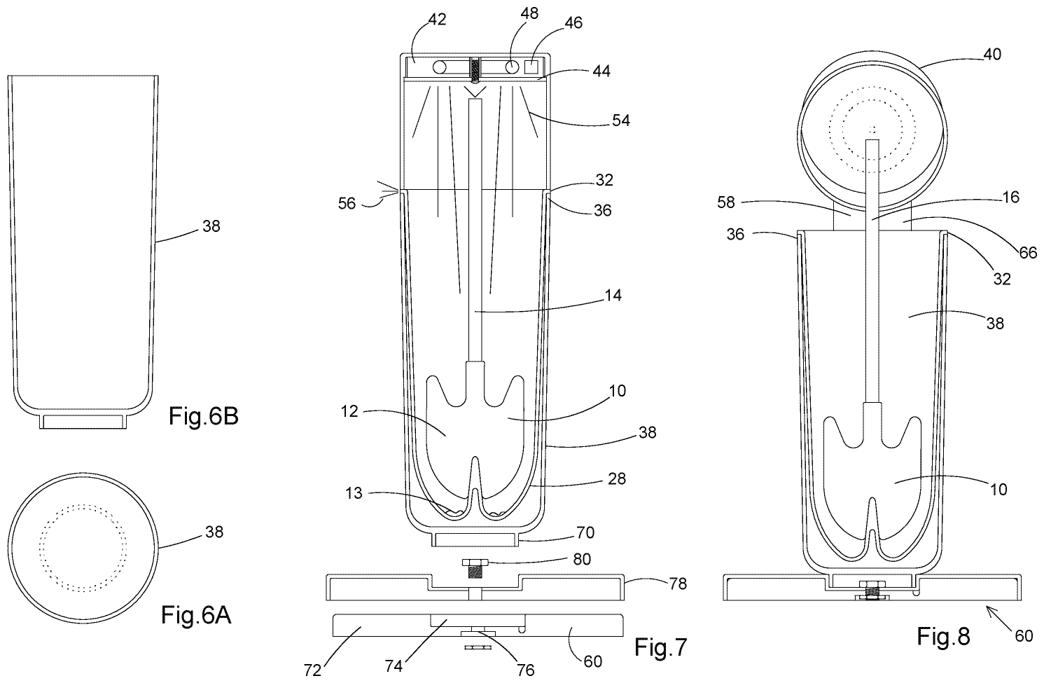

The container 38 and a corresponding lid 40 are separately shown in FIG. 5 and FIG. 6. The container 38 is essentially cylindrical and open towards the upper side. The insert 28 is inserted into the container 38. The container lid 40 is designed with two walls at its upper end, whereby a closed chamber 42 is formed together with a further bottom of quartz glass or borosilicate glass 44 which is transparent for UVC-light. A control 46 and one or more ultraviolet light sources 48 are provided inside such chamber. The ultraviolet light sources 48 can be, for example, an emitting ring or several point-shaped light sources. The light sources are arranged such that the radiation 54 of the UV light sources 48 emit into the inside 42 of the lid 40 and into the inside 50 of the container 38. The side walls and other limitations of the lid 40 and the container 38 are not transparent for UVC radiation. The radiation cannot exit towards the outside.

A portion of the radiation reaches the insert 28. The insert 28 consists of acryl. This material will convert the wavelength which is not visible for the human eye to a longer, visible wavelength. Thereby, the acryl will shine violet or blueish. The rim 32 is visible from the outside. Consequently, a switched-on light source 48 can be recognized from the outside by a shining rim 32. This is illustrated by radiation 56 in FIG. 7. For removing the insert, such as, for example, for cleaning, the container is provided with a handling recess 79. This can be seen in FIG. 11.

FIG. 8 and FIG. 12 show the assembly with open container lid 40. It can be recognized that the handle 16 extends beyond the upper edge 36 of the container 38 and the rim 32 of the insert 28. Thereby, the toilet brush 10 can be easily taken up. Accordingly, the lid 40 is not plane, but cylindrical. The handle 16 extends into the interior of the lid 40 when the lid 40 is closed. This can be well recognized in FIG. 7.

The lid 40 is hinged to the container 38 with a hinge unit 39. The hinge unit 39 comprises a hinge. A contact area 58 is provided at the upper edge of the container side portion of the hinge unit 39. When the lid 40 is closed the contact area 58 hits a sensor connected to the control, the electronic components of which are accommodated by the hinge unit 39. The hinge unit is fixed to the container and the lid.

The sensor detects the contact as a signal indicating that the lid 40 is closed. Then the exposure with UVC radiation is started by the control 46. If the lid 40 is opened the contact to the sensor is interrupted. Then the UV-exposure is interrupted or cannot be activated.

The control 46 is provided with a clock or another timer. In the present embodiment the clock measures the exposure time. When the exposure time is 30 seconds the exposure is finished. It can be assumed that the handle 16, the inner walls of the insert and the rest of the toilet brush 10 is fully disinfected thereafter.

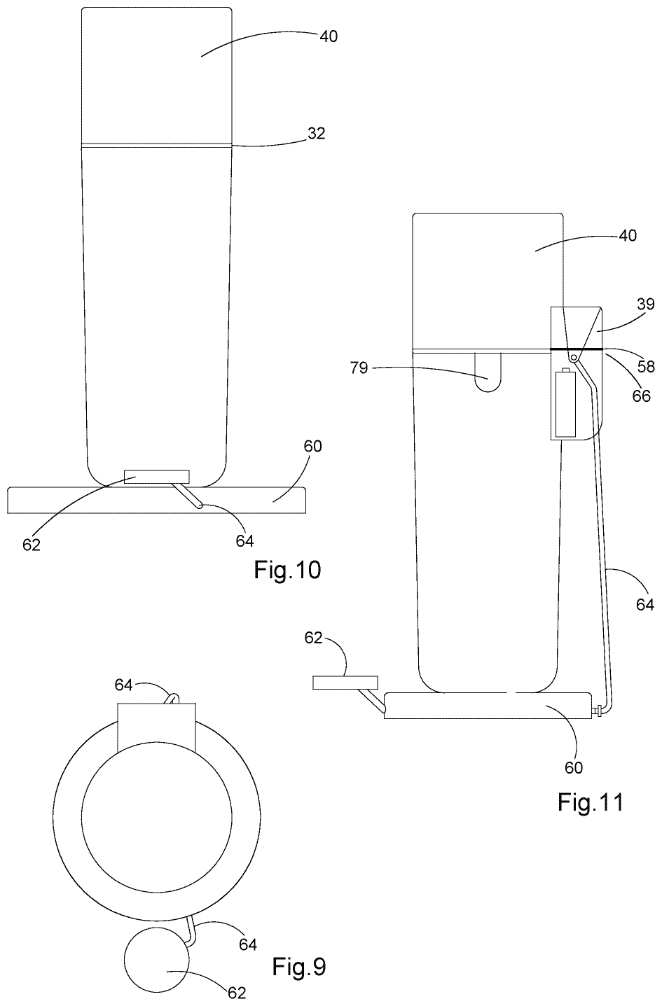

The opening mechanism for the lid 40 is illustrated in FIGS. 9 to 12. The container 38 stands on a base plate 60. For this purpose the container 38 is provided with a downwardly projecting profile 70. This is shown in FIG. 7. In the present embodiment the profile 70 is formed by a ring which is integrated in the underside of the container 38. The base plate 60 is composed of two parts. A plate 72 with high density, such as of stone or stainless steel is provided with a receptacle 74 and a bore hole 76. A cover 78 is set on the plate 72. The cover 78 has the shape of the plate 72 and is also provided with a receptacle. The cover 78 is screwed to the plate with a screw 80. The profile 70 engages with the corresponding profile 74 of the base plate 72. It can then lock-in, be held by a bayonet catch, clipped or fixed in any other way. In such a way a high stability is achieved. At the same time the cover may be made of light material, such as the same material as the container 38.

A pedal 62 is provided at the base plate 60. The pedal 62 is connected to the lid 40 by a system of levers 64. The lid 40 is hinged to the container 38 by a hinge 66. As with a commercially available pedal bin the pedal 62 can be moved by foot from the upper position which is shown in FIG. 10 and FIG. 11 to a lower position which is shown in FIG. 12. The lid 40 is then moved into an open position by the system of levers 64. This is illustrated in FIG. 12. The user must not, therefore, manually touch the lid. The user only takes up the already disinfected handle 16 of the toilet brush 10. After taking it the foot can be removed from the pedal 62. The lid is automatically closed by gravity.

When the pedal returns to its idle position the lid is closed. The then empty container is exposed to radiation. In particular, such ranges are exposed to radiation and disinfected which are otherwise shaded by the brush. This includes, for example, the pin for centring the brush.

After its use the user returns the toilet brush 10 into the container 38. The pedal 62 is also operated by foot for this purpose in order to open the lid 40 again. When the lid 40 is closed again after inserting the toilet brush 10 the contact 58 presses on the sensor. Then the exposure to UV light is started.

FIG. 12 shows the system of levers 64 in greater detail. Contrary to known pedal bins for waste the system of levers upwardly extends outside of the container. The upper end of the system of levers 64 ends at the hinge 66 in the hinge unit 39. Apart from the sensor and the contact area 58 the hinge unit 39 serves to accommodate batteries or accumulators 68. They are accessible from the outside through a flap. The UV lights inside the lid are connected through a simple wire.

The first embodiment is a freely moveable standing housing with batteries or accumulators. It is, however, also possible to use a housing suspended at the wall with a connected power supply, as it is described below with respect to the second embodiment.

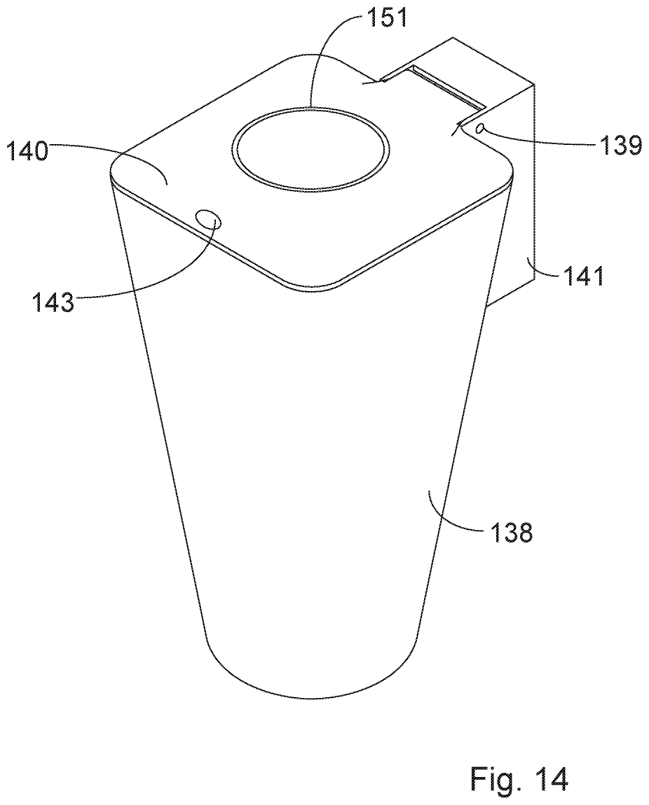

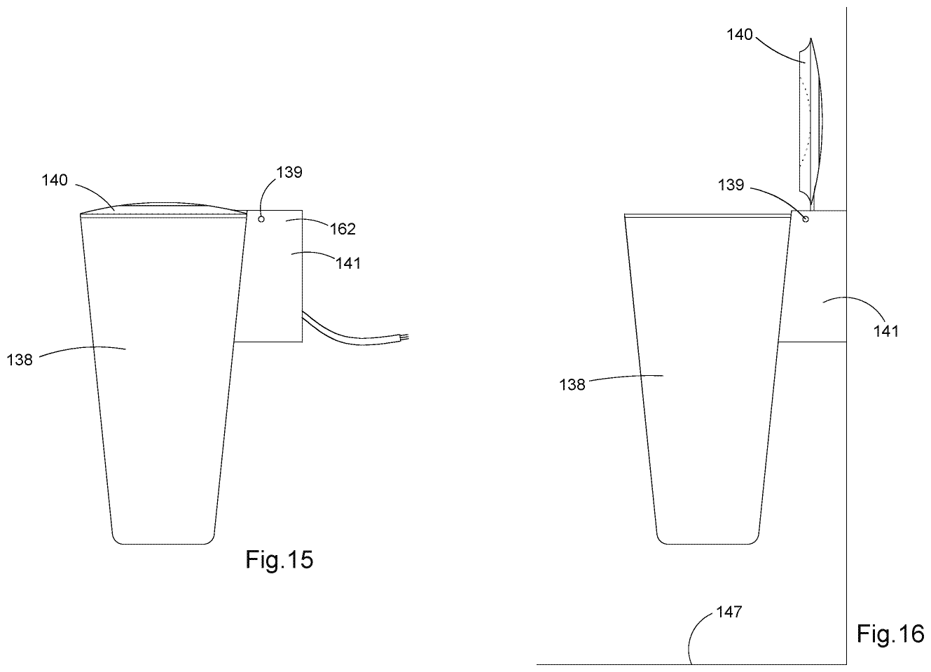

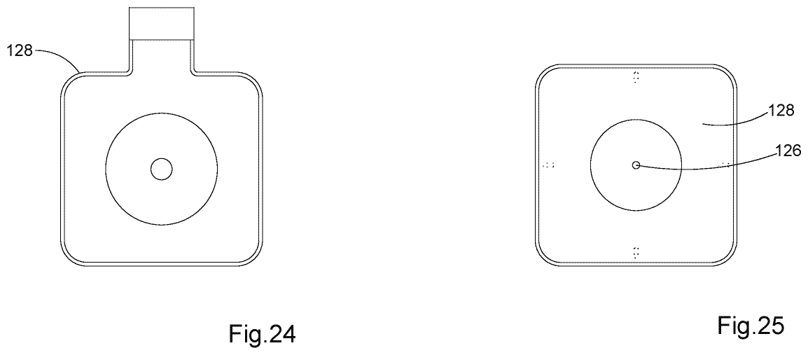

### 2. Embodiment: Housing Suspended at the Wall (FIG. 14 to FIG. 25)

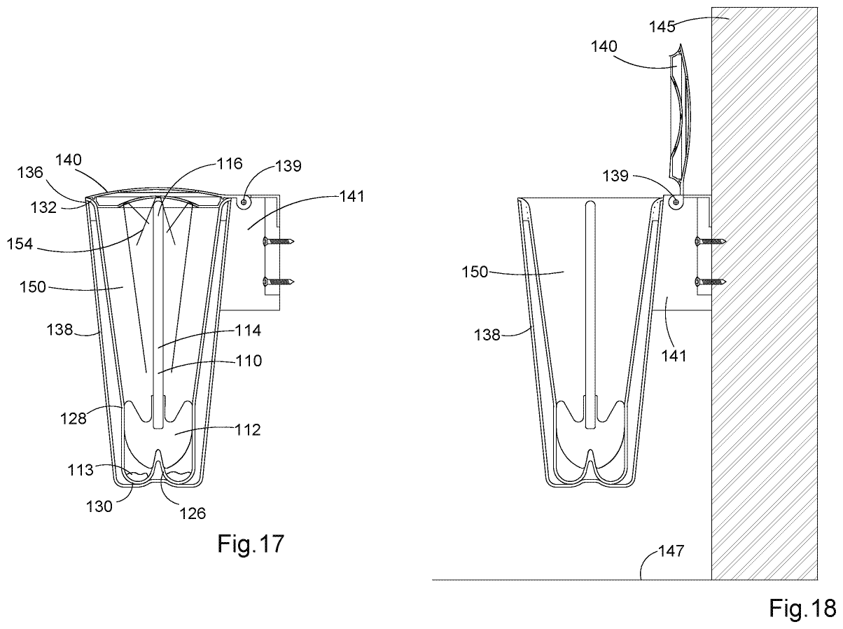

FIG. 17 shows a toilet brush generally designated with numeral 110. The toilet brush 110 has a brush head 112 and a shaft 114. The upper portion 116 of the shaft 114 forms a handle. The toilet brush 110 corresponds to the toilet brush 10 of the first embodiment.

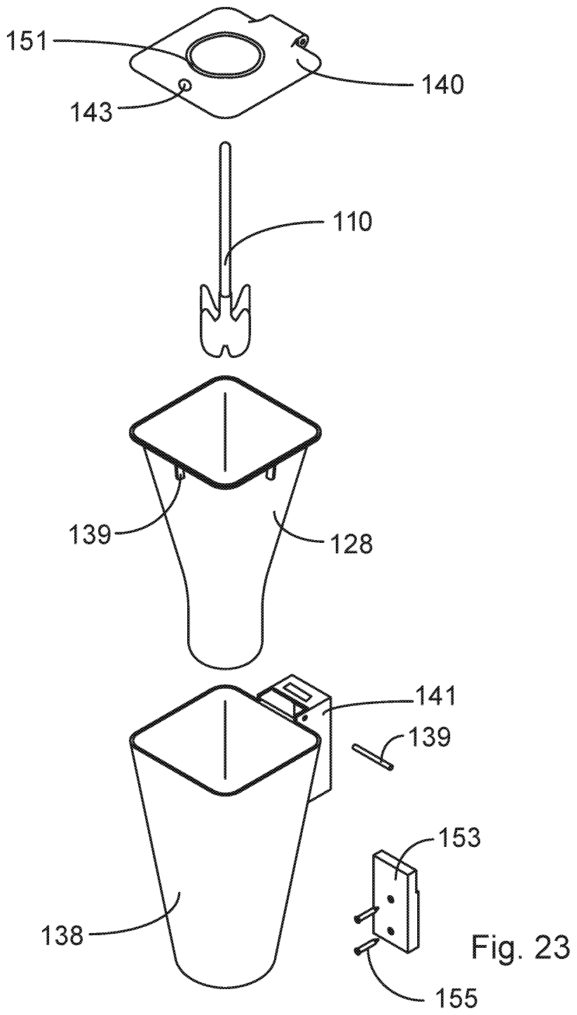

The toilet brush 110 can be stuck onto an upwardly extending pin 126 in an insert 128 and centred in the insert 128. The insert 128 is separately shown in FIG. 23 and FIG. 25.

The insert 128 serves to accommodate the toilet brush 110 when it is not used for cleaning. The insert 128 of the present embodiment consists of plastic. It is understood, that different materials with different or similar characteristics may also be used. The insert 128 has a circular cross section in the bottom range. In the upper range the insert has an essentially square cross section. This can be well recognized in FIG. 23. The bottom of the insert is slightly curved and the pin 126 is formed in such a way that the toilet brush 110 will not fully reach the bottom 130. This can be well recognized in FIG. 17. If dripping water 113 accumulates at the bottom of the insert 128 it is avoided thereby that the toilet brush 110 is unnecessarily contacted therewith.

At its upper end the insert 128 forms an outwardly projecting rim 132. The insert lays on an upper edge 136 of an outer container 138 with such upper rim 136. This can be recognized in FIG. 17 and FIG. 18. Projections 139 form a central part of each side below the rim 136. This can be recognized in FIG. 23. The position of the insert 128 in the container 138 is determined by such projections 139. Therefore, the insert 128 has practically no play in the container 138.

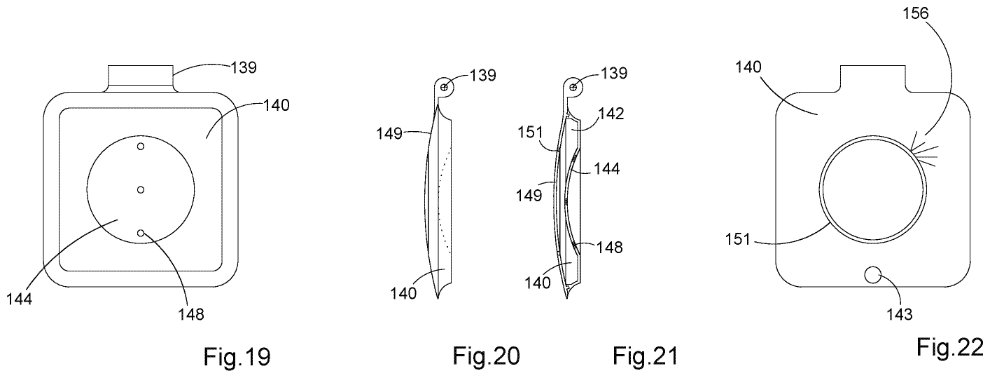

The lid 140 belonging to the container 138 is separately shown again in FIGS. 19 to 22. The container 138 is open at the upper end. In the present embodiment the container is essentially square with rounded corners and the container has a circular cross section in the lower range. The insert 128 is inserted into the container 138.

The container lid 140 has a double bottom with an upper bottom 149 and a lower bottom 144. A closed chamber 142 is formed by the upper bottom 149 and the lower bottom 144. A control and three ultraviolet light sources in the form of LEDs 148 are accommodated in the chamber 142. The upper bottom 149 and the lower bottom 144 are curved and expand towards the outside. The LEDs extend through the lower bottom 144. Thereby, radiation 154 of the LEDs can be emitted into the interior 150 of the container 138. The side walls and other limitations of the lid 140 and of the container 138 are non-transparent for UVC radiation. The radiation cannot exit to the outside.

The curvature of the lid 140 effects different emission directions of the LEDs arranged therein. In such a way the LEDs will emit in the direction of the handle of the toilet brush 110 and in particular in the direction onto the handle 116. This can be well recognized in FIG. 17.

A portion of the radiation is emitted directly into the chamber 142 or slightly upwards by reflection. An acryl ring 151 is integrated in the upper bottom 149 of the lid. This ring can be well recognized in FIG. 22. The material converts the wavelength which is not visible for the human eye to a larger, visible wavelength. Thereby, the acryl will shine slightly violet or blueish. The ring 151 is visible from the outside. Therefore, the shining ring 151 can be seen from the outside indicating that the LEDs 148 are switched on. This is illustrated by radiation 156 in FIG. 22.

FIG. 16 and FIG. 18 show the assembly with an open container lid 140. The container lid 140 is rotatably hinged about an axis 139 at a housing 141, which is connected to the container 138.

A contact area is provided at the upper edge of the housing 141. When the lid 140 is closed the contact area hits a sensor which is connected to the control and which has electronic components accommodated in the housing 141.

The sensor detects the contact in the form of a signal indicating that the lid 140 is closed. Then the exposure with UVC radiation is started by the control. When the lid 140 is opened the contact to the sensor is interrupted. Then the UV exposure is interrupted or cannot be activated.

The control is provided with a clock or another timer. In the present embodiment the clock measures the exposure time. When the exposure time is 30 seconds the exposure is finished. It can be assumed that the handle 116, the inner walls of the insert and the rest of the toilet brush 110 is fully disinfected thereafter.

The lid 140 is opened with an opening mechanism which is accommodated in the housing 141. The opening mechanism comprises a motor 162 and a control which receives the signal of a motion sensor 143. The motion sensor 143 is provided at the upside at the lid 140. If the user approaches the motion sensor 143, with a hand, for example, a signal is transmitted to the control of the motor 162. The motor 162 will then open the lid 140 and the toilet brush can be taken up. When the toilet brush is returned after its use back into the insert, the lid 140 is closed upon manual activation of the motion sensor by the motor 162. The contact will then push onto the sensor at the upper edge and the exposure with UV light will be started. Since the motor 162 is accommodated in the housing 141 the lid can be designed particularly light. Only small forces are then required for opening.

The housing 141 serves to accommodated the hinge 139 and the motor 162 for opening the lid 140. Furthermore, it serves to fix the entire container assembly at a wall 145. This can be well recognized in FIG. 16 and FIG. 18. The wall fixing leaves some free space below the container which enables easy cleaning of the floor 147 in the range of the container.

Screws 155 are used for fixing a fixing means 153 which can be well recognized in FIG. 18. Then the housing 141 is stuck onto the fixing means 153 by means of a guiding.

A wall suspended container 138 is practically immobile. This enables the power supply of the container by connection to a power network rather than by batteries or accumulators. Suitable components such as rectifiers, transformers or the like are accommodated in the housing 141 which adapt the network power in the usual way to the requirements of the motor 162 and the LEDs 148. Nothing must be serviced, re-filled, exchanged or otherwise added. The container 138 or the insert 128, resp. and the brush 110 must only cleaned like any other common toilet brush. This can be carried out also by untrained staff

The above embodiment is fixed before a wall. It is, however, also possible to use a housing flush behind the wall also with connection to a power network as it is described below with respect to the third embodiment.

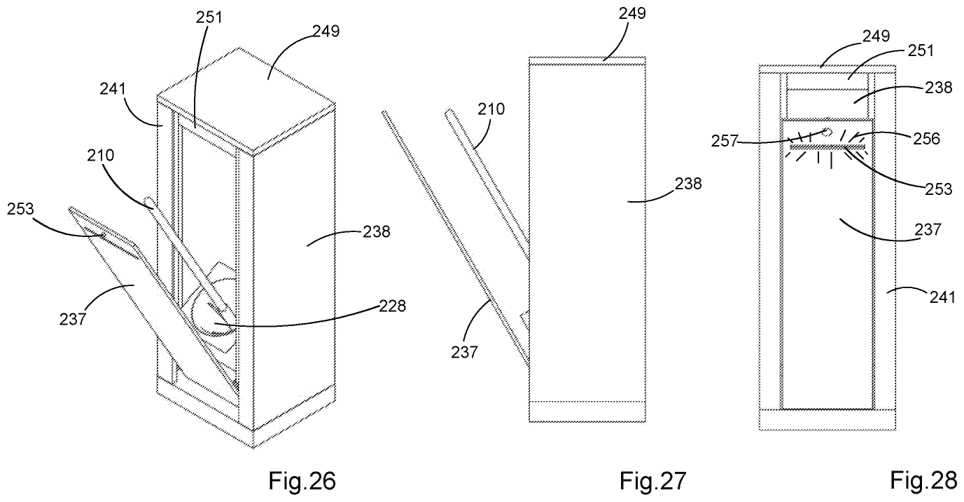

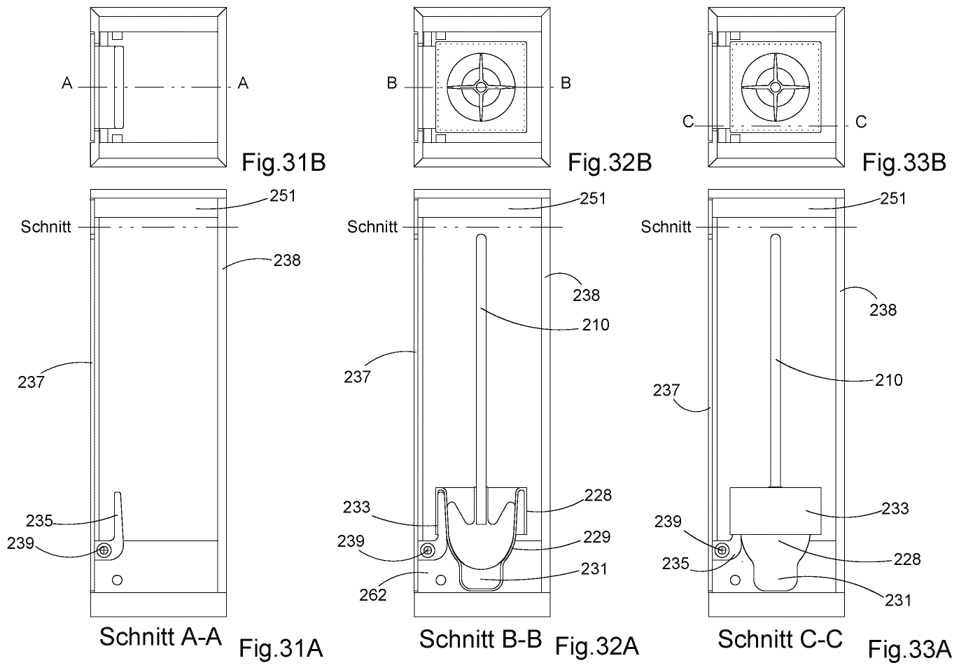

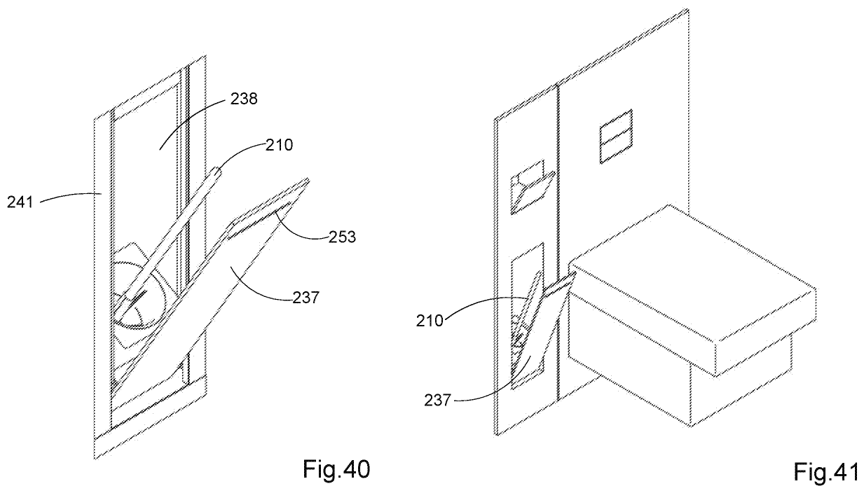

### 3. Embodiment: (FIG. 26 to FIG. 41)

FIG. 36 shows a toilet brush generally designated with numeral 210. The toilet brush 210 has a brush head 212 and a shaft 214. The upper portion 216 of the shaft 214 forms a handle. The toilet brush 210 corresponds to the toilet brush 10 of the first embodiment.

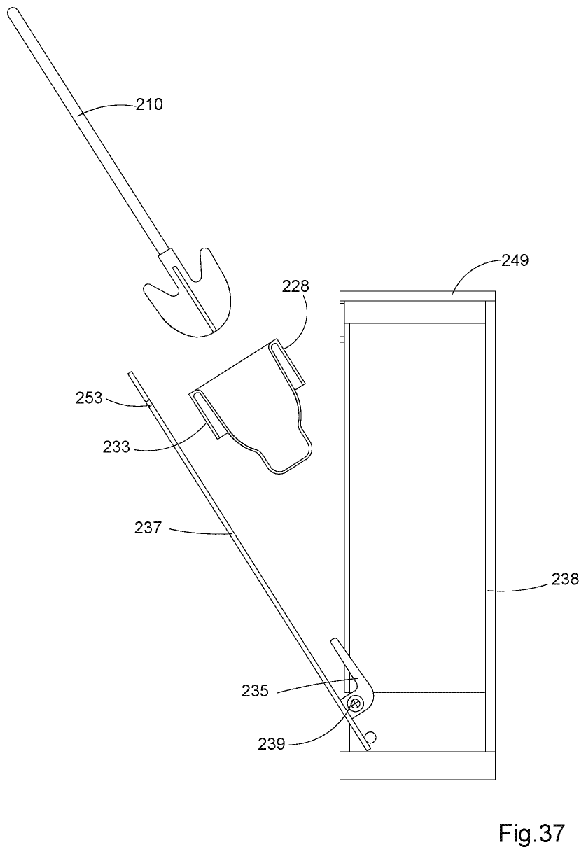

The toilet brush 210 can be stuck into an insert 228 and centred in the insert 228 by the side walls. The insert 228 is separately shown in FIGS. 30 and 37.

The insert 228 serves to accommodate the toilet brush 210 when it is not used for cleaning. The insert 228 of the present embodiment consists of plastic. It is understood, that different materials with different or similar characteristics may also be used. The insert 128 is pot shaped and has a circular cross section at the bottom. The wall of the insert 228 is adapted to the form of the brush head 212. Thereby the brush 210 is supported in its position. The insert 228 forms a downwardly extending recess 231 below the brush head 212. The recess 231 forms a chamber to receive dripping water. Thereby, it is avoided that the toilet brush is unnecessarily contacted with dripping water when it accumulates at the bottom of the insert 228.

The insert 228 extends upwards up to about the upper end of the brush head 212. Consequently, the shaft 214 of the toilet brush is not enclosed by the insert 228. At its upper edge the insert 228 forms a rectangular collar 233. The insert 228 is suspended at a hook 235 with such collar 233.

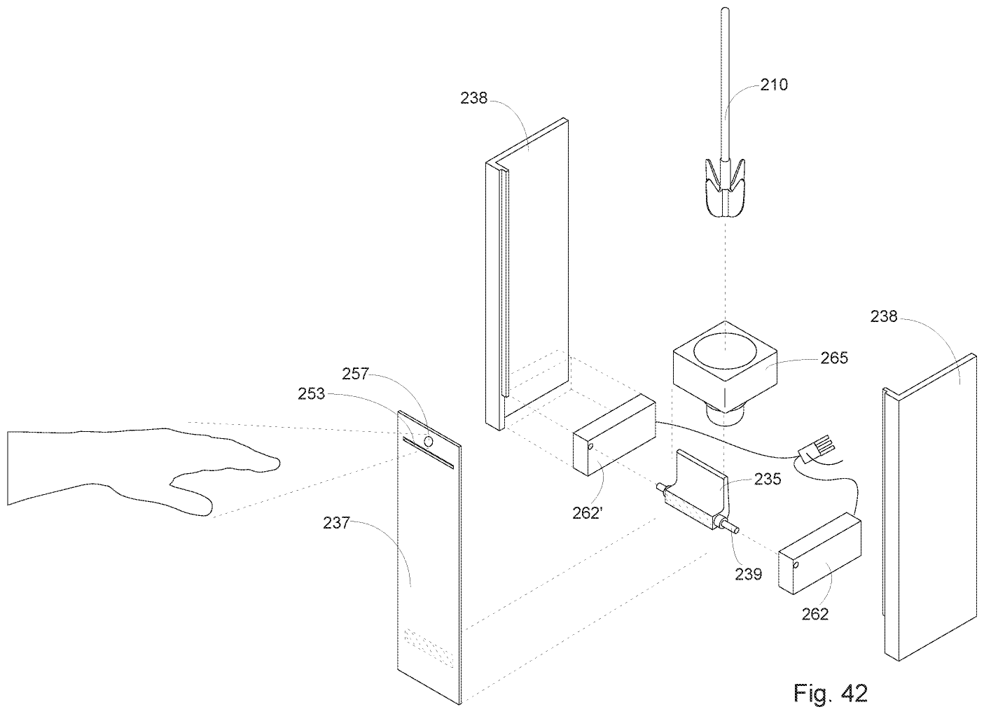

The hook 235 extends along the entire width of a housing flap 237 and is fixed thereto, by, for example gluing. It is also possible to fix the hook 235 in another way or to integrate the hook in the housing flap 237. The housing flap 237 is rotatable about a shaft 239. The shaft 239 is fixed in the lower range of the hook 235 and rotatably held in a recess in the housing 238. At least one end of the shaft is driven by a motor 262. FIG. 42 shows an embodiment with two motors 262 and 262′ driving the shaft 238. If the flap 237 opens or closes the housing 238 the hook 235 and the suspended insert 228 will follow this rotational movement. A toilet brush 210 which is present in the insert 228 will, therefore, be rotated towards the outside. This can be well recognized in FIGS. 26 and 35.

The flap 240 belonging to the housing 238 is separately shown again in FIGS. 19 to 22. The housing 238 is open at the front. In the present embodiment the housing in this range is essentially cuboid. The insert 228 is inserted into the housing 238.

The flap 237 and a lining 241 which is provided on the side of the flap 237 are visible from the front for the user. Accordingly, they may be made of a material which is adapted to the taste of the user, such as glass, wood or stainless steel.

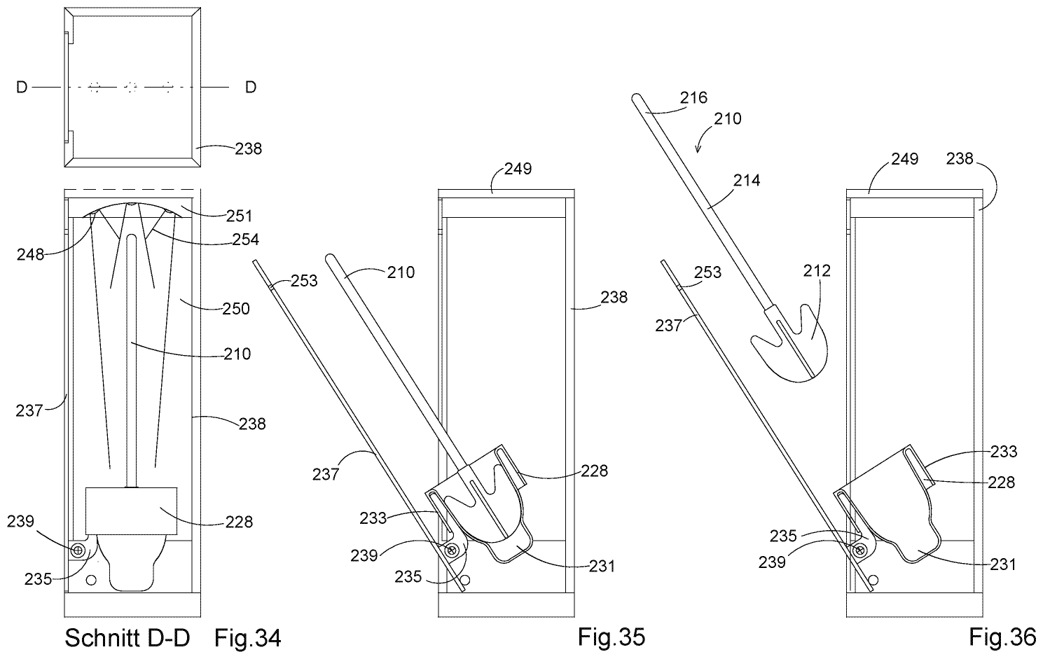

The housing 238 is closed by an upper cover 249. A module 251 with a control and three ultraviolet light sources in the form of LEDs 248 is fixed at the cover 249. This can be seen in FIG. 30. The front of the module 251 is only visible in an open state. The flap is opened there. When the flap is closed the module 251 is not visible from the outside. The front side serves as a stop and is provided with a plastic stopper, sponge rubber or the like which is inserted in a groove or glued on. The underside of the module 251 is curved and bends outwardly. The LEDs extend to the bent underside of the module 251. Thereby radiation 254 can be emitted by the LEDs 248 into the interior 250 of the housing 238. This can be well recognized in FIG. 34. The inner limitations of the flap 237 and the housing 238 are intransparent for UVC radiation. The radiation cannot exit towards the outside.

The curvature of the underside of the module 251 causes different radiation directions of the LEDs 248 arranged therein. In such a way the LEDs emit in the direction of the handle of the toilet brush 210 and in particular in the direction onto the handle 216. This can be well recognized in FIG. 34.

A strip 253 of acryl material is integrated in the upper range of the flap. This can be well recognized in FIG. 28. The material converts wavelengths which are not visible for the human eye to a longer, visible wavelength. Thereby the acryl will shine slightly violet or blueish. The strip 253 is visible from the outside. LEDs 248 which are switched on can be recognized from the outside due to the shining strips 253. This is illustrated by radiation 256 in FIG. 28.

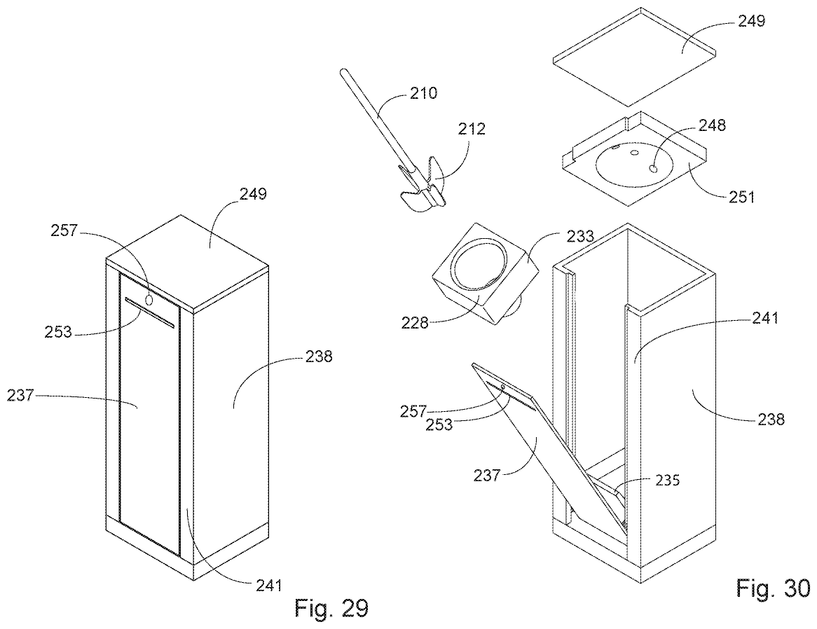

When the housing is closed the front of the flap 237 is aligned with the lining. FIG. 26 and FIG. 27 show the assembly with open flap 237. The flap is rotatable about an axis 239 for opening and closing the housing opening. A motor 262 is used for this purpose. This can be well recognized in FIG. 42. The motor 262 is activated by a motion sensor 257. The motion sensor 257 is located above the strip 253 at the flap 237. When the motion sensor 257 detects a movement, such as by a hand, the flap is opened by the motor 262. The detection of a further movement will cause a closing signal for the motor 262. For damping the contact between the flap 237 and the housing a beading with a sponge rubber is provided at the upper edge. The sensor 257 can, as shown in FIG. 29, be provided above the strip 253. However, it is also possible to position the sensor on the lid, for example, at the front or in the middle. This is particularly useful with installations before the wall. The connection of the sensor and the motor 262 can be wireless, such as by radio or through a wire in a groove.

When the flap 237 is closed the exposure with UVC radiation is started by the control. When the flap 237 is opened the UV exposure is interrupted or cannot be activated.

The control is provided with a clock or another timer. In the present embodiment the clock measures the exposure time. When the exposure time is 30 seconds the exposure is finished. It can be assumed that the handle 216, the inner walls of the insert and the rest of the toilet brush 210 is fully disinfected thereafter.

If the user approaches the motion sensor 257, with a hand, for example, a signal is transmitted to the control of the motor 262. The motor 162 will then open the flap 237 and the toilet brush can be taken up. When the toilet brush is returned after its use back into the insert, the flap 237 is closed upon manual activation of the motion sensor by the motor 262. Then the exposure with UV light will be started.

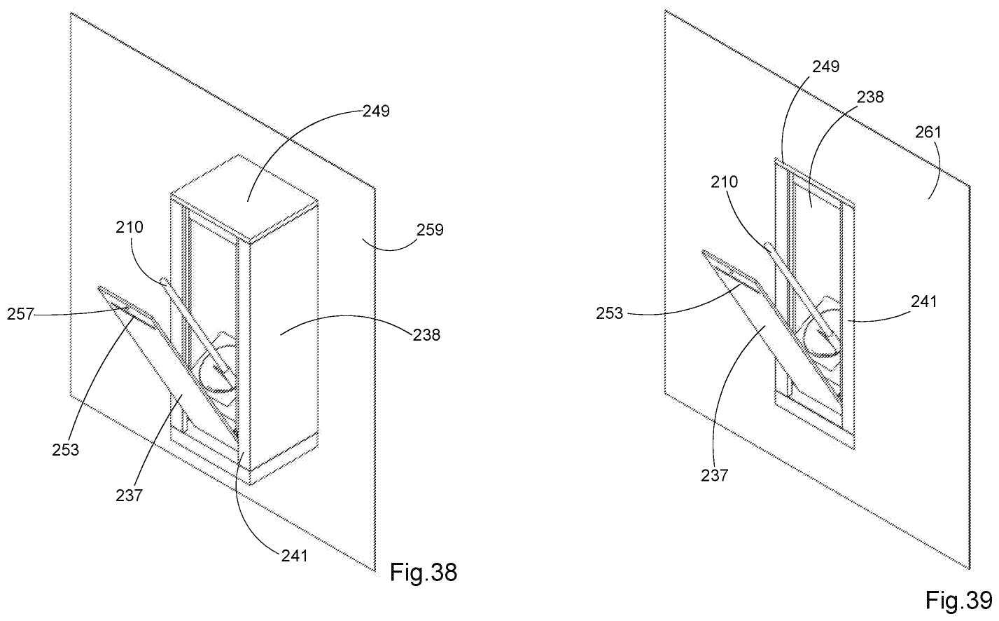

The housing 238 with the toilet brush 210 can be installed before the wall or before a glass plate or any other decorative plate 259, as it is shown in FIG. 38. However, the housing 238 may also be flush-mounted in the wall. This is illustrated in FIG. 39 by way of example. The liner 241 will then be aligned with the flap 237 and the wall 261. Finally, it is also possible to integrate the housing individually or with other installations in a toilet installation with a glass plate or a decorative plate 263. This is illustrated in FIG. 41 by way of example.

A fixedly installed housing 238 is practically immobile. This enables the power supply of the container by connection to a power network rather than by batteries or accumulators. Suitable components such as rectifiers, transformers or the like are accommodated in the housing 238 which adapt the network power in the usual way to the requirements of the motor 262 and the LEDs 248. Nothing must be serviced, re-filled, exchanged or otherwise added. The container 238 or the insert 228, resp. and the brush 210 must only cleaned like any other common toilet brush. This can be carried out also by untrained staff.

The above described embodiments serve to illustrate the invention claimed in the claims only. Features which are disclosed together with other features, generally can be used alone or in combination with further features which are explicitly disclosed in the text or in the drawings or implicitly in the embodiments. Measures and sizes are indicated by way of example only. The person skilled in the art will derive suitable ranges from is expertise and must, therefore not be described in greater detail here. The disclosure of a precise embodiment of a feature does not mean that the invention shall be limited to such precise embodiment. On the contrary: such a feature can be replaced by many others which are known to the person skilled in the art. The invention can, therefore, not only be carried out in the form as described in the embodiment but by all embodiments which are covered by the accompanying claims.

The term “up”, “down”, “right”, and “left” exclusively relate to the accompanying drawings. It is understood that the claimed devices can also assume a different orientation. The terms “comprising” and “including” mean that further not-mentioned components may be provided. The terms “essentially”, “mostly”, or “mainly” relate to all features which have a property or a content with a majority, i.e. more than all other components or features, such as with two components, for example, more than 50%.

1. An assembly for storing and disinfecting toilet brushes comprising
a toilet brush having a shaft, a handle and a brush head;
a container or a housing for accommodating the entire toilet brush, wherein said container or said housing is provided with an opening for taking up said toilet brush;
a lid, a flap or another closing portion for closing said opening;
a UV-light source emitting UV-light having a wavelength range and an intensity,
said UV-light source provided for exposing at least said handle of said toilet brush to said UV-light, and
wavelength range and said intensity selected such that the UV-light is suitable for the disinfection of parts of said toilet brush; and wherein
opening means are provided for opening said container or said housing without hand contact; and
control means are provided for controlling said UV-light source in such a way that said UV-light source emits UV-light only when said container or said housing is closed.

2. The assembly of claim 1, and wherein said container or said housing is provided with a releasable insert for said toilet brush.

3. The assembly of claim 2, and wherein said toilet brush has a profile and said insert is provided with a bottom having a profile adapted to receive said toilet brush with a corresponding profile.

4. The assembly of claim 1, and wherein said container has a lid or a housing cover and said UV-light source and said control means are accommodated in said container lid or below said housing cover.

5. The assembly of claim 1, and wherein said control means comprise a timer for determining the an exposure time and said UV light source is switched off after a set exposure time.

6. The assembly of claim 1, and wherein said container has a lid and a sensor is provided for detecting a position of said lid, said sensor generating signals which are transmitted to said control means.

7. The assembly of claim 6, and wherein said sensor is provided with a pin switch, a contact with book cover function or another contact, detecting when said container is closed so that said control means generate a signal for starting said UV light.

8. The assembly of claim 1, and wherein said container is provided with a rim, hook or projection and said insert is provided with a radially outwardly projecting upper rim or a collar holding said insert at said rim, hook or projection in said container.

9. The assembly of claim 1, and wherein said housing forms an opening which is closed by parts of said insert or another element which can be at least partly seen from outside even with said lid being closed and which is made of acryl or another material which will colourfully shine upon exposure to said UV-light source.

10. The assembly of claim 1, and wherein said lid is connected to a pedal which is adapted to move said lid, said flap or other closing element to an open position by foot.

11. The assembly of claim 1, and wherein a motor is provided for moving said lid, said flap or said closing element.

12. The assembly of claim 11, and wherein said motor for moving said lid, said flap or said closing element receives control signals from a motion sensor.

13. The assembly of claim 1, and wherein said housing is adapted to be flush-mounted and said flap is aligned with a wall or a front side of an installation with said housing installed therebehind.
